(19) United States
(12) Patent Application Publication
(10) Pub. No.: US 2024/0304316 A1
(43) Pub. Date: Sep. 12, 2024

(54) ARTIFICIAL INTELLIGENCE-BASED SYSTEMS AND METHODS FOR DETECTING ANOMALOUS DATA

(71) Applicant: MASTERCARD INTERNATIONAL INCORPORATED, Purchase, NY (US)

(72) Inventors: Shawn Mehrhoff, Saint Ann, MO (US); Christopher T. Scholl, Saint Peter's, MO (US); David Vorhies, Saint Charles, MO (US); James W. Barkhurst, Fenton, MO (US)

(21) Appl. No.: 18/661,407

(22) Filed: May 10, 2024

Related U.S. Application Data
(63) Continuation-in-part of application No. 16/696,217, filed on Nov. 26, 2019, now abandoned.

(51) Int. Cl. G16H 40/20; G06N 20/00; G06Q 40/02

(52) U.S. Cl. CPC G16H 40/20; G06N 20/00; G06Q 40/02

(57) ABSTRACT

A computer system for analyzing data using AI modeling tools to detect anomalous data associated with a person. The computer system includes at least one processor and an AI model. The at least one processor is programmed to receive person data associated with the person including payment transaction records, input the person data into the at least one AI model to generate one or more outputs including (i) identifying anomalous data of the person, (ii) identifying at least one present need of the person from the anomalous data, and (iii) outputting a recommendation for addressing the at least one present need and transmit a notification message to a caregiver computer.

### CROSS-REFERENCE TO RELATED APPLICATIONS

This application is a continuation-in-part and claims priority to U.S. patent application Ser. No. 16/696,217, filed Nov. 26, 2019, the entire contents and disclosures of which are hereby incorporated herein by reference in its entirety.

### FIELD OF THE DISCLOSURE

The field of the present disclosure relates generally to analyzing data using artificial intelligence (AI) tools, and more particularly to AI-based systems and methods for analyzing captured data associated with a person to detect anomalous data, identifying a present need of the person associated with the anomalous data, and identifying a service provider to address the identified need of the person.

### BACKGROUND

In some known cases, a person may experience difficulty performing or completing various activities associated with daily living, such as shopping for groceries, filling prescriptions, scheduling and attending appointments, maintaining a residence (e.g., lawn care, home repairs, etc.), and/or selecting and hiring a service provider (e.g., a plumber, a home repair service person, an electrician, etc.). The person may suffer from an illness or condition that impedes the ability of the person to complete these activities, for example, the person may have limited mobility, and/or memory disorders, etc. The person may wish to hire a service provider to help complete certain activities or tasks; however, the person may be apprehensive about choosing a service provider. The person may wish to hire the best service provider (e.g., least expensive, most reliable, most experienced, etc.). It may be especially difficult to select the best service provider if there are multiple service providers to choose from. In some cases, the person may wish to hire a service provider they previously hired but is unable to remember which service provider it was.

In some cases, a caregiver may be responsible for monitoring the needs of the person and/or helping the person complete activities. In some cases, the caregiver may be unable to visit the person on a frequent or regular basis. For example, the caregiver may have prior time commitments, such as full or part-time employment. In other cases, the caregiver may live a substantial distance away from the person making it time consuming for the caregiver to visit the person. In these types of cases, it may be difficult for the caregiver to confirm and/or monitor the needs of the person and to assess whether the person is completing specific tasks. For example, the caregiver may be unable to confirm that the person is paying bills on time, attending doctors' appointments, and/or taking prescribed medications. As such, the caregiver may attempt to monitor the needs of the person by periodically checking in with the person (e.g., calling or texting), to confirm that the person has completed specific tasks and/or to monitor the needs of the person. In some cases, the person may be hesitant and/or resistant to conveying their needs to the caregiver. Additionally, a caregiver may wish to aid the person in selecting and hiring a service provider to address a need of the person but may also be apprehensive in selecting the best service provider from a list of service providers.

It may be advantageous to have AI-based methods and systems which help a caregiver take care of a person by constantly monitoring certain data associated with the person, detecting anomalous data from the monitored data, identifying a need or needs of the person from the data, alerting the caregiver of the need(s), and recommending a service provider which may be hired to address the need(s) of the person.

### BRIEF DESCRIPTION

In one aspect, a computer system for analyzing data using AI modeling tools to detect anomalous data of a person is provided. The computer system includes at least one memory device, an AI modeling component for storing at least one model configured to identify anomalous data of the person wherein the at least one model is trained using historical user transaction data for a plurality of users that is labeled with user need data, and at least one processor in communication with the at least one processor and the AI modeling component. The at least one processor being programmed to receive person data associated with the person including payment transaction records including at least an account identifier associated with a payment account of the person and a merchant identifier for identifying a merchant involved in the transaction. The at least one processor is further programmed to input the person data into the at least one AI model to generate one or more outputs including (i) identifying anomalous data of the person, (ii) identifying at least one present need of the person from the anomalous data, and (iii) outputting a recommendation for addressing the at least one present need and transmit a notification message to a caregiver computer device associated with a caregiver of the person. The notification message including the identified at least one present need of the person and the outputted recommendation.

In another aspect, a computer-implemented method for analyzing data using AI modeling tools to detect anomalous data of a person is provided. The method implemented using a computer device including at least one memory device for storing data, and an AI modeling component for storing at least one model configured to identify anomalous data of the person wherein the at least one model is trained using historical user transaction data for a plurality of users that is labeled with user need data. The at least one processor is in communication with the at least one memory and the AI modeling component. The method includes receiving person data associated with the person including payment transaction records including at least an account identifier associated with a payment account of the person and a merchant identifier for identifying a merchant involved in the transaction. The method includes inputting the person data into the at least one AI model to generate one or more outputs including (i) identifying anomalous data of the person, (ii) identifying at least one present need of the person from the anomalous data, and (iii) outputting a recommendation for addressing the at least one present need and transmitting a notification message to a caregiver computer device associated with a caregiver of the person. The notification message including the identified at least one present need of the person and the outputted recommendation.

In a further aspect, a non-transitory computer-readable storage medium that includes computer-executable instructions for analyzing data using AI modeling tools to detect anomalous data of a person is provided. The computer-executable instructions executed by a data optimization computer device including at least one memory device, an AI modeling component for storing at least one model configured to identify anomalous data of the person wherein the at least one model is trained using historical user transaction data for a plurality of users that is labeled with user need data, and at least one processor in communication with the at least one memory and the AI modeling component. When the instructions are executed, the processor is configured to receive person data associated with the person including payment transaction records including at least an account identifier associated with a payment account of the person and a merchant identifier for identifying a merchant involved in the transaction. The processor is further configured to input the person data into the at least one AI model to generate one or more outputs including (i) identifying anomalous data of the person, (ii) identifying at least one present need of the person from the anomalous data, and (iii) outputting a recommendation for addressing the at least one present need and transmit a notification message to a caregiver computer device associated with a caregiver of the person. The notification message including the identified at least one present need of the person and the outputted recommendation.

In one aspect, a computer system for aiding a caregiver in monitoring the needs of a person is provided. The computer system includes at least one processor in communication with a transaction database and a service provider database. The transaction database stores a plurality of transactions records associated with a plurality of transactions each initiated by one of a plurality of account holders at one of a plurality of merchants. Each transaction record includes a merchant identifier associated with the respective merchant and an account identifier associated with the respective account holder, and at least one of the account holders is the person. The service provider database stores a plurality of service provider records each associated with one of a plurality of service providers. Each service provider record includes a service provider identifier, a service provider contact data, and a service provider description associated with the types of goods and/or service provided by the service provider. The service provider description is associated with a need of the person. The at least one processor is configured to monitor a plurality of transaction parameters of the person, including monitoring the transaction database for transaction records including the account identifier associated with the person and determine if at least one of the plurality of transaction parameters being monitored satisfies a transaction parameter alert criteria. In response to at least one of the transaction parameters satisfying the transaction parameter alert criteria, the at least one processor determines a first need of the person. The at least one processor then queries the service provider database to retrieve a first service provider record having the service provider description associated with the first need of the person. The at least on processor also transmits a referral message to the caregiver. The referral message including the first need of the person and identifies the service provider associated with the first service provider record.

In another aspect, a computer-implemented method for aiding a caregiver in monitoring the needs of a person is provided. The method implemented using a computing device including a processor in communication with a memory device for storing data The method includes a transaction database storing a plurality of transactions records associated with a plurality of transactions each initiated by one of a plurality of account holders at one of a plurality of merchants. Each transaction record includes a merchant identifier associated with the respective merchant and an account identifier associated with the respective account holder. At least one of the account holders is the person. The method also includes a service provider database storing a plurality of service provider records each associated with one of a plurality of service providers. Each service provider record includes a service provider identifier, a service provider contact data, and a service provider description associated with the types of goods and/or service provided by the service provider. The service provider description is associated with a need of the person. The at least one processor is in communication with the transaction database and the service provider database. The at least one processor is configured to monitoring a plurality of transaction parameters of the person, including monitoring the transaction database for transaction records including the account identifier associated with the person and determining if at least one of the plurality of transaction parameters being monitored satisfies a transaction parameter alert criteria. In response to at least one of the transaction parameters satisfying the transaction parameter alert criteria, the at least one processor determines a first need of the person. The at least one processor also queries the service provider database to retrieve a first service provider record having the service provider description associated with the first need of the person. The at least one processor also transmits a referral message to the caregiver. The referral message includes the first need of the person and identifies the service provider associated with the first service provider record.

In a further aspect, a non-transitory computer-readable storage medium that includes computer-executable instructions for aiding a caregiver in monitoring the needs of a person is provided. The non-transitory computer-readable storage medium includes a transaction database, a service provider database, and at least one processor in communication with the transaction database and the service provider database. The transaction database stores a plurality of transactions records associated with a plurality of transactions each initiated by one of a plurality of account holders at one of a plurality of merchants. Each transaction record includes a merchant identifier associated with the respective merchant and an account identifier associated with the respective account holder. At least one of the account holders is the person. The service provider database storing a plurality of service provider records each associated with one of a plurality of service providers. Each service provider record includes a service provider identifier, a service provider contact data, and a service provider description associated with the types of goods and/or service provided by the service provider. The service provider description is associated with a need of the person. The at least one processor is configured to monitor a plurality of transaction parameters of the person, including monitoring the transaction database for transaction records including the account identifier associated with the person and determining if at least one of the plurality of transaction parameters being monitored satisfies a transaction parameter alert criteria. In response to at least one of the transaction parameters satisfying the transaction parameter alert criteria, the at least one processor determines a first need of the person. The at least one processor queries the service provider database to retrieve a first service provider record having the service provider description associated with the first need of the person. Also, the at least one processor transmits a referral message to the caregiver. The referral message includes the first need of the person and identifies the service provider associated with the first service provider record.

### BRIEF DESCRIPTION OF THE DRAWINGS

FIGS. 1-6 show example embodiments of the methods and systems described herein.

FIG. 1 is a simplified schematic diagram of an example process flow between components of a caregiving system including a caregiving computer device for constantly monitoring data associated with a person, applying AI tools, identifying the needs of the person and/or identifying a service provider that may be hired by the person or a caregiver to assist with the need.

FIG. 2 is a data flow diagram illustrating an example process implemented using the caregiving system shown in FIG. 1.

FIG. 3 is a process flow diagram of an example of a caregiving process for monitoring the needs of a person and identifying a service provider.

FIG. 4 is another process flow diagram of an example of a caregiving process for monitoring the needs of a person and identifying a service provider.

FIG. 5 illustrates an example configuration of the caregiving system computer device shown in FIG. 1.

FIG. 6 illustrates an example configuration of a person computer device and/or the caregiver computer device shown in FIG. 1.

### DETAILED DESCRIPTION

The systems and methods described herein are directed to analyzing data using artificial intelligence (AI) tools, and more specifically to an AI-based system and method for analyzing captured data associated with a person to detect anomalous data associated with the person, identifying a present need of the person associated with the anomalous data, alerting a caregiver to the need, and identifying a service provider to address the identified need of the person. In some cases, the person (e.g., a person with impairments related to old age, disability, and/or disorders, etc.) may rely on the caregiver (e.g., family member, caretaker, nurse, guard, etc.) for assistance in completing various tasks associated with activities of daily living. In some cases, the caregiver may have a substantial role in the care of the person and as such, the caregiver may monitor one or more parameters associated with the person on a frequent or daily basis in order to determine the needs of the person. In other cases, the person may need limited assistance; however, the person and/or the caregiver may wish to have the peace of mind confirming that the needs of the person are being addressed.

The system and processes described herein include transmitting and receiving a plurality of messages between the person, the caregiver, a caregiving system, and a payment processing network. The caregiving system may be communicatively connected to a payment transaction database, a service provider database, a personal care database, and/or AI modeling tools. The transaction database includes a set of transaction records each associated with a corresponding one of a plurality of transactions by one of a plurality of accountholders with one of a plurality of merchants. Each transaction record includes a transaction identifier (ID) associated with the respective transaction, an account number associated with the respective accountholder, a merchant identifier (ID) associated with the respective merchant, and other data that is captured as part of payment transaction processed over a payment network. For example, each of the transaction records may also include transaction data including a date/time of the transaction, a transaction amount, and/or a description of the goods or services purchased in the transaction. In other words, the transaction database includes information regarding historical payment transactions between the plurality of accountholders and the plurality of merchants. The payment processing network may build the transaction database by extracting data from payment transaction messages routed over the payment processing network, building transaction records from the extracted data, and storing the transaction records to the transaction database.

One example of such a payment processing network is the Mastercard® Interface Processor (“MIP”), which provides a gateway to, and message handling infrastructure for communications on, the Mastercard® payment network. Alternatively, the system may include a payment aggregator/payment gateway, an ACH system, a banking system, a Fintech system, and/or other network processor or “switch” systems. Those of skill in the art will appreciate that such proprietary communications standards used by each payment network may be variations of a standardized format, such as ISO 8583 or ISO 20022 compliant messages. As used herein, “ISO” refers to a series of standards approved by the International Organization for Standardization (ISO is a registered trademark of the International Organization for Standardization of Geneva, Switzerland). The ISO 8583 and ISO 20022 standard defines acceptable message types, data element locations, and data element values. In addition, the ISO 8583 standard reserves several data element locations for private use.

The caregiving system may be associated with a caregiving system computer device. The caregiving computer device may retrieve at least a subset of transaction records from the transaction database. The caregiving system computer device may determine a service provider that meets a need of the person from a set of merchants based on data contained in the set of transaction records. The service provider may be selected based on predefined metrics, e.g., a proximity of the merchant to the person, the most commonly used merchant, the cheapest merchant, the merchant with the least amount of “negative” transactions (e.g., chargebacks), and/or online reviews such as social media reviews. In some embodiments, the caregiving system computer device may sort the transaction records by the type of merchant in order to categorize the merchants based on the types of goods and/or service provided or sold by the merchant. For example, a set of merchants may be categorized into, for example and without limitation, a plumbing service category, a landscaping category, a home repair category, etc. Each merchant category may include one or more merchants.

The caregiving computer device is configured to determine a scoring parameter for one or more predefined metrics, for each merchant contained in each merchant category, based on transaction data contained in the transaction records. The scoring parameter may be used to select a service provider from a plurality of merchants within a merchant category. The scoring parameter may be based on, for example and without limitation, the proximity of the merchant, the number of transactions occurring with the merchant, a number of negative transactions (e.g., refunds, chargebacks), the average amount per transaction, and/or any combination of these. In other words, the caregiving system computer device may use information contained in the transaction database to determine, from a set of merchants, service providers that best satisfy the one or more predefined metrics.

The caregiving system may build the service provider database by storing a plurality of service provider records to the service provider database. Specifically, the service provider database includes a set of service provider records, with each record corresponding to one of the plurality of merchants. Each service provider record includes a merchant description and service provider contact information. The merchant description includes a description of the goods and/or service provided or sold by the service provider.

In some embodiments, one or both of the caregiver and the person may enroll (e.g., register) with the caregiving system. During registration, the person and/or the caregiver may provide personal details associated with the person to the caregiving system. Personal details may include, for example and without limitation, the person's age, weight, medical conditions, medications, and/or personal calendar, etc. The registration process may also capture details about the caregiver, such as the caregiver's calendar. In some embodiments, registration includes an authorization for the caregiving system to track or monitor a plurality of parameters associated with the person. In this example embodiment, registration may authorize the caregiving system to track and monitor payment transactions initiated by the person. The caregiving system computer device may generate a personal care record for the person based on data received during registration or subsequent updates submitted by the person or the caregiver. The caregiving system computer device may build the personal care database by storing a plurality of personal care records to the personal care database.

The system and method described herein may include certain opt-in requirements in order for persons, caregivers, cardholders, merchants, acquirers, and/or issuers to participate within the system. For example, a cardholder or person using the user computing device to make purchases in combination with their assigned PAN may enroll as a participating cardholder/person in the system for allowing their PAN to be used as part of the model training and/or as input in the model to determine whether the person has a particular need based upon the analyzed data. In addition, as part of registering with the system, a person, a caregiver, an issuer, a merchant and/or an acquirer may be given the option to opt-in to the system for using certain AI tools and/or models to determine whether the person has anomalous data and a particular need. Enrollment within the system may include acceptance of certain service terms, preferred contact information (e.g., email, SMS text notification, push notification, notification via a digital wallet service, etc.) and preferences for service notifications and the like, or other desired information relating to the cardholder to provide the services. In contemplated embodiments, the enrollment includes opt-in informed consent of users to data usage by the system consistent with consumer protection laws and privacy regulations. In some embodiments, the enrollment data and/or other collected data may be anonymized and/or aggregated prior to receipt such that no personally identifiable information (PII) is received. In other embodiments, the system may be configured to receive enrollment data and/or other collected data that is not yet anonymized and/or aggregated, and thus may be configured to anonymize and aggregate the data. In such embodiments, any PII received by the system is received and processed in an encrypted format, or is received with the consent of the individual with which the PII is associated. In situations in which the systems discussed herein collect personal information about individuals including persons, caregivers, cardholders or merchants, or may make use of such personal information, the individuals may be provided with an opportunity to control whether such information is collected or to control whether and/or how such information is used. In addition, certain data may be processed in one or more ways before it is stored or used, so that personally identifiable information is removed.

In some embodiments, the caregiving system may continuously or periodically monitor (e.g., by sampling, measuring, evaluating) parameters (e.g., a tracking parameter and/or a transaction parameter) associated with the needs and/or activities of the person. The caregiving system may continuously or periodically monitor a transaction parameter associated with payment transactions initiated by the person by continuously or periodically retrieving or monitoring payment transactions for the person that are processed by the payment network. The person may initiate a purchase transaction (“payment transaction”) by providing payment credentials (e.g., a credit or debit card number, a bank account number, user login information corresponding to saved payment credentials, digital wallet information, etc.) to a merchant for the exchange of goods and/or services. After the person initiates a purchase transaction, the payment processing network typically stores a transaction record in the transaction database as discussed above. In some examples, the payment processing network may identify, during processing of the payment transaction, the payment credentials as belonging to the person registered with the caregiving system and separately transmit the transaction record to the caregiving system computer device.

Alternatively, the caregiving computer device may monitor (e.g., periodically query) the transaction database for new transaction records associated with the person. In response to receiving or retrieving the personal transaction record, the caregiving system may transmit a notification message to the caregiver indicating that the person has initiated a payment transaction. In some example embodiments, the caregiving system may determine if a transaction parameter contained in the personal transaction record satisfies a transaction parameter alert criteria. For example, the caregiver may wish to confirm that the person has attended an appointment, such as a doctor's appointment, with a health care service provider. While attending the appointment, the person may initiate a payment transaction, for example a copayment, with the healthcare service provider. The transaction record associated with the person's payment credentials and the healthcare service provider may trigger the caregiving system (and the AI component) to transmit a notification message to the caregiver indicating that the person has attended the appointment.

In some embodiments, the caregiving system computer device may continuously or periodically monitor a tracking parameter associated with the person. The caregiving system may receive and/or retrieve a plurality of tracking messages, including the tracking parameter, from one or more devices associated with the person. Devices associated with the person may include, for example and without limitation, a mobile device, a computer device, a medical device, and/or a biometric device. For example, the caregiving system may be linked to a wearable biometric device, e.g., Fitbit, step counting device, and/or heart rate monitor. Additionally, or alternatively, the caregiving system may be linked to a medical device of the person, for example and without limitation, a pacemaker, a heart monitor, and/or a glucose monitoring system. The tracking parameter associated with the person may include, for example and without limitation, a biometric parameter of the person, such as weight, heart rate, steps taken, blood sugar levels, etc. Additionally, or alternatively, the tracking parameter may include Global Positioning System (GPS) data that may be used to determine the location of the person.

In certain embodiments, the caregiving system computer device determines if the tracking parameter satisfies a tracking parameter alert criteria. If the tracking parameter satisfies the tracking parameter alert criteria, the caregiving system computer device may determine a need of the person and/or transmit a notification message to the caregiver. For example, the caregiving system computer device may receive a plurality of tracking messages from a glucose monitoring device of the person. The tracking messages including the tracking parameter of blood sugar levels associated with the person. The caregiving system computer device may set a tracking parameter alert criteria to include a blood sugar level varying outside an accepted range. As such, if the blood sugar satisfies the tracking parameter alert criteria, then the caregiving system computer device transmits a notification message to the caregiver indicating the blood sugar level of the person.

In some embodiments, either one or both the person and/or the caregiver are communicatively coupled to the caregiving system, such that the person and/or the caregiver may transmit and/or receive a plurality of messages with the caregiving system. For example, either one or both of the caregiver and/or the person may transmit a query message and/or a request message to the caregiving system. The query message may be associated with asking for a recommendation for a service provider. For example, the person may need assistance cleaning the gutters of their residence. The person and/or the caregiver may transmit a query message to the caregiving system computer device, requesting a recommendation for a gutter cleaning service provider.

The caregiving system computer device may then retrieve a service provider record from the service provider database, for example by applying one or more predefined metrics as discussed above, for a service provider associated with a gutter cleaning service. The request message may be associated with the tracking or monitoring of a tracking parameter and/or a transaction parameter of the person. For example, the caregiver may transmit a request message to the caregiving system requesting to be notified if the person initiates a purchase transaction with specific merchants. For example, the caregiver may wish to know when the person is traveling and may request to be notified when a payment transaction is initiated with a transportation service provider. In other words, one of the caregiver and/or the person may request to set the transaction parameter alert criteria and/or the tracking parameter alert criteria.

The caregiving system computer device may also be in communication with an AI modeling component that generates one or more model outputs when the caregiving system computer device applies one or more model inputs. Model inputs may include data associated with either the person and/or the caregiver. For example, model inputs may include the person's transaction data (referred to herein as “person transaction data”) for transactions having an account identifier associated with the person. Person transaction data may include transaction amounts, merchants, merchant category codes (MCC), items purchased, average amount of transactions, recurring payment data (e.g., frequency of recurring payments and/or amounts of recurring payments), transaction location, transaction dates/times, and the like. Model inputs may also include additional and/or alternative data of the person, e.g., medical history, medications, demographic data, calendar data and/or sensor data gathered from sensors strategically placed within the home of the person or worn by the person. The calendar data may be supported by a computer device of the person, e.g., scheduling or calendar applications executable on the computer device of the person. In some embodiments, model inputs may include sensor data collected by the computer device of the person or other sensors around or worn by the person, for example, sensor data may include location data, e.g., collected by a global positioning sensor (GPS) application executable on the computer device of the person. Additionally, and/or alternatively, sensor data may include telematics data, velocity or acceleration data, gyroscopic data, and the like.

In some embodiments, model inputs may include the caregiver selected transaction parameter alert criteria and/or tracking parameter alert criteria. In some embodiments, the caregiving system computer device may utilize the transaction parameter alert criteria and/or the tracking parameter alert criteria in training data used to train the model, e.g., a person specific model. Additionally, and/or alternatively, caregiving system computer device may use the transaction parameter alert criteria and/or the tracking parameter alert criteria to filter and/or restrict model outputs.

Model inputs and/or model training data may also include group data (e.g., data that is indirectly related to the person and/or the caregiver). Group data may refer to any data associated with a group of a plurality of people, for example, a group may refer to a demographic group having members, which may include the person, having similar demographics. Group data may further include timing data such as holidays (e.g., federally recognized holidays) or events (e.g., Superbowl or other sporting events), and/or time of year and/or seasons that may impact or effect transaction data. Group data may also include financial transactions data of the group, for example and without limitation, local or regional average monthly expenditures, average expenditures per transaction, average expenditures per transaction for a particular MCC, etc.

Model outputs may include the identified need of the person. Model outputs may include messages to be transmitted to either, or both, of computer devices associated with the person and/or the caregiver. In some embodiments, the model outputs may include an identification of a service provider enabled to address the identified need of the person. In some embodiments, the model output includes a plurality of identified service providers each enabled to address the identified need of the person. In some embodiments, the model output may include a ranking for each of the plurality of service providers based on one or more metrics determined by the caregiving model, also referred to herein as an AI model. Metrics may include user ratings, when or if the service provider was previously hired by the person, cost, and/or service provider location relative to the location of the person. The model output may also include computer code that is executable by a user device to cause certain data to be displayed on the user device. Such data may include reporting interfaces or dashboards and/or selectable input mechanisms (e.g., virtual buttons, etc.) that may be selected by a user (person or caregiver) for selecting a service provider or scheduling service provider with a single click after the recommendation has been presented.

The caregiving system computer device may generate the model using any suitable techniques, e.g., train, tune, and/or re-trains the model using machine learning methodologies and/or artificial intelligence techniques. In some embodiments, the caregiving system computer device may build one or more training datasets that the caregiving system computer device may use to train or tune the model.

In some embodiments, the caregiving system computer device may build a first training dataset and train the model in a first training session. The first training dataset may include a plurality of historical records each associated with a plurality of different historical users. Each historical record may include historical transaction data, historical user data, and/or historical sensor data. Each historical record may include a historical identified need of the user. The historically identified need of the person may be an identified need as determined by a historical caregiver of the historical users. Additionally, and/or alternatively, the historically identified need of the person may have been previously identified by the model and/or confirmed as a need of the person by the person and/or the caregiver.

In some embodiments, the caregiving system computer device may build a second training dataset including a plurality of person specific historical records each associated with a single person. The caregiving system computer device may re-train or generate a user specific model, using the second training dataset. For example, the caregiving system computer device may train, in a first session, the model using the first training dataset, and then subsequently retrain or tune the model using the second training dataset to generate the user specific model. In some cases, the training data set includes labeling data that helps to train the model to recognize relationships between data such as between transaction data of a user and a known need of the user. In other words, the labeling data may help to identify the purpose(s) of certain purchases made by historical users.

In some embodiments, the caregiving system computer device may train the model in a first session using both the first and second training datasets. In some embodiments, the caregiving system computer device may train the model using the first and second training datasets using weighting factors to more heavily influence the second training dataset, e.g., the weighting factors of the second training dataset are greater than weighting factors of the first training dataset.

In some embodiments, the caregiving system computer device trains and or re-trains a person specific model using person data to generate a person profile. The person profile may include the person specific model and/or model outputs which are representative of the normal and/or acceptable behavior of the person. The caregiving system computer device may utilize the person profile, and received current person data, to determine a need of the person. For example, the caregiving system computer device may compare the person profile to current person data to detect changes in the persons behavior and/or normal or acceptable financial transaction data to determine the need of the person. Additionally, and/or alternatively, the caregiving system computer device may apply current person data as model inputs to the person specific model to generate model outputs including the need of the person.

In some embodiments, the caregiving system computer device generates one or more notification messages. The caregiving system computer device made transmit the notification messages to the caregiver computer device associated with the caregiver. The notification message may include one or more model outputs. In some embodiments, the notification message is a model output. The notification message includes the identified need of the person and/or the identified service provider.

The caregiving system may include a caregiving escalation system, wherein the caregiving system computer device determines a severity level for the identified need of the person. The severity level may be determined as a model output of the model. Additionally, and/or alternatively, the severity level may be based on, at least in part, the transaction parameter alert criteria and/or the tracking parameter alert criteria. For lower ranked identified needs of the person, the caregiving system computer device may transmit one or more prompting messages to the person computer device, prompting the person to respond to the prompting message, remind the person to complete one or more tasks, or prompting the person to hire and/or purchase the identified service provider. In other words, in some embodiments, for low risk identified needs of the person, the caregiving system computer device transmits prompting messages directly to the person computer device, reducing the emotional load on caregivers and/or data transmission volumes. For moderately and/or high ranked identified needs of the person, the caregiving system computer device may transmit a notification message to the caregiver computer device and a prompting message to the person computer device. In some embodiments, the caregiving system computer device may transmit notification messages only to the caregiving system computer device.

In some embodiments, the caregiving escalation system may also transmit notification messages to the caregiver computer device based on one or more response messages from the person computer device. In some embodiments, the caregiving system may apply person response messages to the model and/or may compare the person response messages to acceptable or known responses. For example, the caregiving system computer device may transmit a prompting message to the caregiving system computer device prompting the person to reply ‘y’ for yes, or ‘n’ for no if the person will be attending an appointment. If the person computer device transmits a response message not including a ‘y’ or ‘n,’ for example, the response message says, “what appointment?” the caregiving system computer device may transmit a notification message to the caregiver computer device, notifying the caregiver that the person is perhaps confused or does not remember their appointment. Alternatively, if the response included a ‘y,’ for example, the caregiving system computer may not transmit a notification message to the caregiver computer device. In some embodiments, the caregiving system computer device may compare person response messages to the person profile to determine acceptable and/or normal responses of the person.

In some embodiments, the caregiving system includes a voicebot executable on person computer device and/or caregiver computer device. The voicebot is communicatively coupled to the caregiving system computer device, such that the caregiving system computer device may receive messages generated by the voicebot from the person computer device and/or caregiver computer device and/or transmit one or more messages to be played over a speaker of the person computer device and/or the caregiver computer device.

Caregiving system computer device may determine parameters of a payment account of the person, e.g., the caregiving system computer device may determine a balance contained within the payment account of the person. In some embodiments, the caregiving system computer device may utilize the balance as a model input. In some embodiments, the caregiving system computer device compares the balance to a person profile and/or model outputs, to determine if the person has sufficient funds to address the need of the person. For example, caregiving system computer device may compare the balance to a recurring payment amount, prior to the scheduled payment, to determine if the person has sufficient funds for the recurring payment. In some embodiments, if the caregiving system computer device determines that the balance is insufficient, the caregiving system computer device may transmit a request message to the caregiver computer device, notifying the caregiver and/or requesting the caregiver to cover the difference between the balance and the recurring payment. In some embodiments, the caregiver may provide an automatic transfer amount, which will transfer from a payment account of the caregiver to a payment account of the person, if one or more criteria is satisfied.

In some embodiments, the caregiving system computer device may apply model inputs including calendar data of the person, sensor data including the GPS location of the person, and/or recent transaction data of the person to the caregiving model to determine if the person is attending a scheduled appointment. In some embodiments, the caregiving system computer device may generate one or more reminder messages to either the person computer device and/or the caregiver computer device, reminding the person to attend an appointment scheduled on the calendar of the person. The reminder message may include a prompt, requesting that the person respond to the message to indicate they are aware of the appointment and/or plan to attend the appointment. The caregiving computer device may apply received person response messages to the model to determine if a notification message should be sent to the caregiver computer device.

As described herein the AI modeling component may include one or more large language models (LLM), such as GPT (Generative Pre-trained Transformers) models, and one or more supplemental models that are configured to curate data from internal and external sources to send to the one or more GPT models for either training the GPT models or inputting the curated data to an already trained model for generating an output. The one or more supplemental models are configured to leverage the one or more GPT models for their wide range of capabilities. In some embodiments, the GPT models may be trained to identify issues or needs for a person based on payment transaction data of the person, group data of other persons, and/or sensor data gathered for the person. For example, one or more of the GPT model may be executed to analyze captured data associated with a person to detect anomalous data of the person, identify a present need of the person associated with the anomalous data, alert a caregiver to the need, and identify a service provider to address the identified need of the person.

In some cases, the AI modeling component is trained using historical labeled transaction data for a plurality of accountholders. In other words, historical transaction data for other persons having known needs may be used to train the AI models. This training data may include seasonal and other timing data along with demographic data of the persons to train the model to identify unusual, anomalous, or otherwise out of trend purchase data for a person, and identify the need of the person based on the anomalous purchase data. Accordingly, the trained model can receive input data that will generate an output of what a person's need is at the present moment, and a recommendation on how to address the need including whether a service provider is need to address the need.

At least one technical problem to be solved by the systems and methods provided herein includes: (i) ability of a remote caregiver to objectively determine a well-being of a person, (ii) ability of a remote caregiver to identify needs of a person, (iii) ability to timely notify the caregiver of the need of the person, (iv) ability to remotely select a quality service provider for the person; and/or (v) ability to analyze transaction data using an AI model to identify anomalous data of a person and identify a need of the person from the anomalous data so as to then recommend a solution to address the need. These problems can be solved using the methods and systems described herein, and can be detected by continuously monitoring transaction data of a person along with sensor data to help further refine the predicted need and solution.

The technical effect of the systems and processes described herein may achieved by performing at least one of the following steps: (i) receive person data associated with the person including payment transaction records including at least an account identifier associated with a payment account of the person and a merchant identifier for identifying the merchant involved in the transaction; (ii) input the person data into the at least one AI model to generate one or more outputs including (i) identifying anomalous data of the person, (ii) identifying at least one present need of the person, and (iii) outputting a recommendation for addressing the at least one present need and (iii) transmit a notification message to a caregiver computer device associated with a caregiver of the person, the notification message including the identified at least one present need of the person and the outputted recommendation.

A technical effect or improvement provided by the systems and processes described herein include at least one of: (i) enabling a remote caregiver to objectively determine a well-being of a person, including monitoring and evaluating transaction records initiated by the person, (ii) facilitating the caregiver in identifying a need of the person, (iii) enabling a remote caregiver to objectively identify an appropriate service provider from a list of merchants; (iv) recommending a service provider that may be hired by the caregiver and/or the person to address a need of the person; (v) reduce data transmission and/or capacity of caregiver using an escalation system.

As used herein, a processor may include any programmable system including systems using micro-controllers, reduced instruction set circuits (RISC), application specific integrated circuits (ASICs), logic circuits, and any other circuits or processor capable of executing the functions described herein.

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are example only, and thus are not limiting as to the types of memory usable for saving of a computer.

In one embodiment, a computer program is provided, and the program is embodied on a computer readable medium. In an example embodiment, the data optimization system is executed on a single computer system, without requiring a connection to a server computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computer devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

As used herein, the terms “transaction card,” “financial transaction card,” and “payment card” refer to any suitable transaction card, such as a credit card, a debit card, a prepaid card, a charge card, a membership card, a promotional card, a frequent flyer card, an identification card, a prepaid card, a gift card, a card that is part of a digital wallet, and/or any other device that may hold payment account information, such as mobile phones, smartphones, personal digital assistants (PDAs), key fobs, and/or computers. Each type of transaction card can be used as a method of payment for performing a transaction. As used herein, the term “payment account” is used generally to refer to the underlying account associated with the transaction card.

The following detailed description illustrates embodiments of the disclosure by way of example and not by way of limitation. It is contemplated that the disclosure has general application to processing financial transaction data by a third party in industrial, commercial, and residential applications.

As used herein, an element or step recited in the singular and proceeded with the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

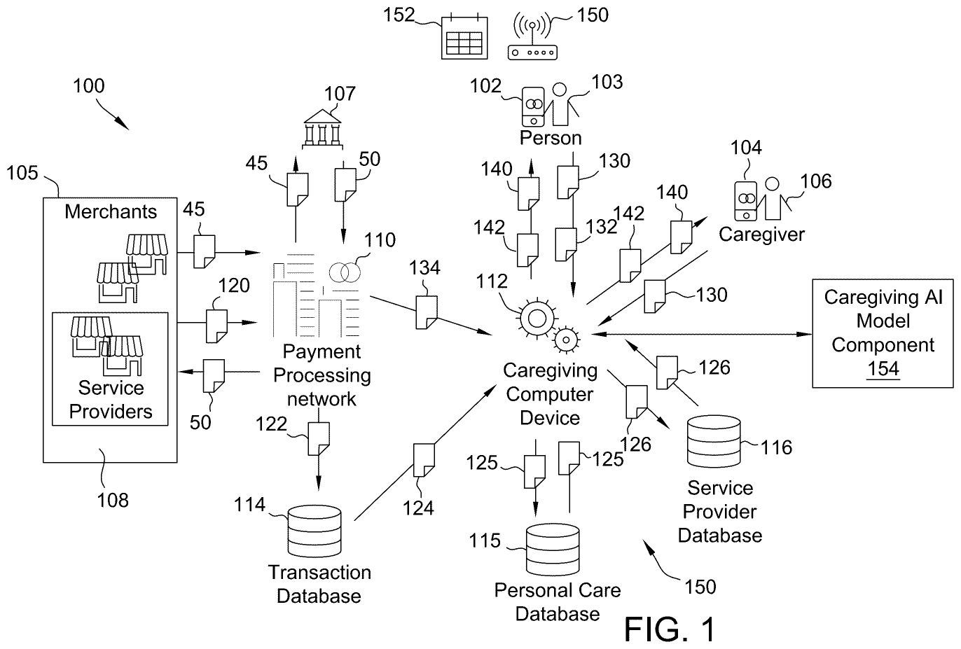

FIG. 1 is a schematic diagram of a caregiving system 100 for monitoring a plurality of parameters of a person 103, determining a need of person 103, and identifying a service provider 108 to meet the determined need in accordance with the present disclosure. In this example, the caregiving system 100, including the caregiving system computer device 112 exchanges exchange of a plurality of messages between person computer device 102 (e.g., a dependent and/or a patient), a caregiver computer device 104, a plurality of merchant computer devices 105, a payment processing network 110. In some example embodiments, caregiving system computer device 112 may be integrated with payment processing network 110. In other embodiments, caregiving system computer device 112 may be separate from, but communicatively connected to a transaction database 114 associated with and/or otherwise configured to receive transaction records 122 from, payment processing network 110.

Payment processing network 110 includes a set of proprietary communications standards for the exchange of financial transaction data and the settlement of funds between financial institutions. Payment processing network 110 is configured to process payment/purchase transactions for a plurality of cardholders (such as person 103) with plurality of merchants 105, by transmitting various authorization request and response messages between parties to the transaction (e.g., the respective merchant 105, an acquirer bank (not shown) associated with the merchant 105, and an issuer 107 of a payment card of the respective cardholder). As used herein, “payment processing network” refers broadly to the network and/or to one or more computer devices associated therewith (e.g., payment processors or payment processing computer devices). Payment processing network 110 may store a merchant description for each merchant 105. The merchant description includes descriptions of the types of good and/or service provided or sold by merchants 105. In some cases, merchants 105 may register with caregiving system 100 by transmitting a description of merchant 105 in a merchant message 120 to caregiving system 100. In some examples, merchants 105 may not be required to register with caregiving system 100 and/or caregiving system 100 may determine the merchant description using other methods. For example, payment processing network 110 may determine the type of goods and service sold or provider by merchants 105 using a merchant category code previously assigned to merchant 105, and this information may be provided to caregiving system 100. The merchant category code is used to distinguish merchants 105 by the type of goods and/or service sold and/or provided by merchants 105.

Upon initiation of a purchase transaction, e.g., by person 103, merchant 105 submits an authorization request message 45 for the transaction through payment processing network 110 to issuer 107 (also known as an issuer bank) for transaction authorization. Issuer 107 processes the request message 45, e.g., by determining whether the cardholder's account is in good standing and has available credit to cover the transaction amount and transmits an authorization response message 50 back through the payment processing network 110 to merchant 105. Authorization response message 50 indicates either denial or approval of the purchase transaction.

In the example, payment processing network 110 is communicatively coupled to transaction database 114 such that the payment processing network 110 may transmit and/or store a transaction record 122 to transaction database 114 in order to create and/or build transaction database 114. In other words, transaction database 114 contains set of transaction records 122. Each transaction record 122 is associated with a corresponding payment/purchase transaction by one of a plurality of cardholders with one of merchants 105. More specifically, each transaction record 122 includes a transaction identifier (ID) associated with the respective transaction, an account number associated with the respective account holder, and a merchant identifier (ID) associated with merchant 105. Each of transaction record 122 may also include transaction data, for example and without limitation, a date/time of the transaction, a transaction amount, and/or a description of the goods or services purchased and/or provided in the transaction. In other words, transaction database 114 includes information regarding payment transactions occurring with a plurality of merchants 105 with a plurality of account holders. At least one of the account holders is the person 103. For merchants registered with caregiving system 100, transaction record 122 may further include the merchant description 120. Alternatively, caregiving system 100 may match transaction record 122 with the corresponding merchant description 120 based on the merchant ID.

Caregiving system computer device 112 is communicatively coupled to transaction database 114. In some examples, payment processing network 110 provides caregiving system computer device 112 access to transaction database 114 maintained by payment processing network 110. Alternatively, payment processing network 110 transmits transaction records 122 to caregiving system computer device 112, and caregiving system computer device 112 adds transaction records 122 to transaction database 114 maintained by caregiving system computer device 112. Accordingly, caregiving system computer device 112 may receive and/or retrieve a subset of transaction records 124 from transaction database 114. Subset of transaction records 124 may be selected and/or filtered from transaction records 122 stored in transaction database 114 using a filtering criteria. The filtering criteria may be determined from the merchant description associated with transaction record 122. In other words, caregiving system computer device 112 may retrieve/receive subset of transaction records 124 from transaction database 114 based on a parameter associated with merchant 105, in order to limit or restrict the number of transaction records 122 being received and/or retrieved from transaction database 114. For example, the filtering criteria may be based on the geographical location of merchant 105. For example, subset of transaction records 124 may exclusively include merchants 105 having a location within a 30-mile radius from a residential location associated with person 103 in personal care database 115. Additionally, or alternatively, the filtering criteria may be based on the types of goods and services provided or sold by merchants 105. For example, subset of transaction records 124 may exclusively include merchants 105 that perform lawn care services, i.e., landscaping service providers, lawn mowing services etc. In other examples, the subset of transaction records 124 may exclusively include merchants 105 that perform plumbing services.

Caregiving system computer device 112 analyzes subset of transaction records 124 in order to identify a service provider 108 from among merchants 105 included within subset of transaction records 124. Service provider 108 may be selected by applying predefined metrics to merchants 105 included within the subset of transaction records 124. In some embodiments, service provider 108 may be associated with the most commonly hired merchant, the least expensive merchant, and/or the merchant with the least amount of chargebacks. For example, the predefined metrics may include one or more scoring parameters. The scoring parameter may be based on, at least in part, data contained in subset of transaction records 124. In other words, a scoring parameter will be determined for each merchant 105 contained within subset of transaction records 124.

For example, subset of transaction records 124 may be filtered to include a set of merchants 105 associated with a landscaping or lawn care service. The predefined metrics selected by the person 103 and/or the caregiver 106 may be a landscaping service provider which charges the least amount for a mowing service. As such, the scoring parameter may be based on the average transaction amount per transaction occurring with the set of merchants 105 associated with a landscaping or lawn care service. Additionally, or alternatively, the predefined metrics selected by the person 103 and/or the caregiver 106 may be a landscaping service provider that is the most commonly used for a mowing service. As such, the scoring parameter may be based on a total number of transactions occurring with the set of merchants 105 associated with a landscaping or lawn care service, over a period of time. In other examples, the predefined metrics selected by the person 103 and/or the caregiver 106 may be whether the service provider 108 was previously hired by person 103. As such, the scoring parameter may be based on transactions occurring with the set of merchants 105 associated with a landscaping or lawn care service that also include the payment credentials of the person 103. In other words, the scoring parameter may be used to determine if person 103 had previously hired merchant 105. Additionally, or alternatively, the predefined metrics and/or the scoring parameter may include a combination of determined parameters. For example, the predefined metrics may be to select five merchants 105 having the five lowest amounts charged based on subset of transaction records 124, and then to select the most commonly used merchant 105 from among the five as the service provider 108.

In response to caregiving system computer device 112 identifying service provider 108, caregiving system computer device 112 may transmit/store a service provider record 126 in service provider database 116. Service provider record 126 includes data associated with service provider 108 to facilitate identification of a previously selected service provider 108 when the corresponding need of the person 103 arises again. In some examples, service provider record 126 includes a look-up index. The look-up index may be associated with the types of goods and/or services provided or sold by service provider 108. Caregiving system computer device may use the look-up index to look-up (i.e., retrieve) service provider record 126 for service provider 108 in response to a subsequent need of person 103.

In other words, caregiving system computer device 112 determines a scoring parameter for each merchant 105 contained in the subset of transaction records 124 retrieved from the transaction database 114. The scoring parameter may be used to identify service provider 108 from the set of merchants 105. Each of service providers 108 is associated with one or more of the needs of the person. For example, service provider database 116 contains a plurality of service provider records 126, wherein each service provider record 126 includes a merchant description that can be used to determine the types of goods and/or services that the corresponding service provider 108 sells or provides. The look-up index may be used to retrieve service provider 108 from service provider database 116 based on the need of person 103.

Person 103 and/or caregiver 106 may register with caregiving system 100 during a registration process by transmitting one or more registration messages 130 to the caregiving system computer device 112. In other embodiments, person 103 and caregiver 106 may transmit registration message 130 to payment processing network 110 in order to register with caregiving system 100. Registration message 130 may include a set of information associated with person 103 and/or caregiver 106. The set of information may include parameters specific to person 103, such as age, weight, location of residency, personal calendar (i.e., appointments, medication schedules, etc.), medical information including dietary restrictions, medications, allergies, etc. The set of information may include alternative or additional parameters associated with person 103. Further, in some examples, registration message 130 includes authorization to track a set of parameters of person 103. For example, registration message 130 may authorize the caregiving system 100 to track the payment transactions initiated by person 103, i.e., authorize access by caregiving system 100 to transaction records 122 including an account number associated with person 103 (i.e., personal transaction records 134).

In some embodiments, caregiving system computer device 112 is communicatively coupled to a personal care database 115. The personal care database may store a plurality of personal care records 125. Each personal care record 125 includes a receiver identifier associated with one of a plurality of care receivers (such as person 103), an item identifier associated with one of a plurality of goods or services required by the respective care receiver, and a schedule data identifying a schedule on which the respective good or service is required. Caregiving system computer device 112 may generate personal care records 125 based on data contained in registration messages 130. Additionally, or alternatively, caregiving system computer device 112 may generate personal care records 125 using data obtained from other sources, e.g., medial charts transmitted from a healthcare service provider. The caregiving system computer device 112 may retrieve one or more personal care records 125 from personal care database 115. In some embodiments, personal care database 115 may be filtered based on a filtering criteria in order to decrease or limit the total number of personal care records 125 that will be considered in a given database operation.

In some embodiments, caregiving system computer device 112 is communicatively coupled a person computer device 102 associated with person 103103 and/or a caregiver computing device associated with caregiver 106. As such, caregiving system computer device 112 may receive and/or retrieve a plurality of a tracking message 132 from the one or more devices associated with person 103 and/or caregiver 106. The computer devices may include a mobile computer device, such as a computer, a laptop, and/or a mobile phone associated with person 103 and/or caregiver 106. In other examples, caregiving system computer device 112 may be connected to other devices associated with person 103. For example, caregiving system computer device 112 may be communicatively connected to, for example and without limitation, one or more sensors 150 associated with, or supported by, person computer device 102. Person computer device 102 may refer to cellphone, a laptop, a tablet, a desktop computer, and the like. In some embodiments, person computer device 102 may include additional and/or alternative devices, such as a wearable biometric tracking device (e.g., Fitbit, fitness watch, step counter, scale, etc.) and/or a medical device or implant (e.g., pacemaker, heartrate monitor, blood pressure machine, and/or wearable glucose monitoring system). Tracking message 132 includes a set of tracking parameters detected by sensors 150 of the person computer device 102 and associated with the person 103. For example, caregiving system computer device 112 may track a set of tracking parameters associated with a biometric of person 103 based on data received/retrieved from the wearable biometric tracking devices. For another example, caregiving system computer device 112 may retrieve tracking message 132 from a mobile computer device associated with person 103, wherein tracking message 132 includes a tracking parameter associated with the geographical location of the mobile computer device 102. For example, in some embodiments, the caregiver 106 and/or the caregiving AI model component 154 may set an alert criteria based on a geographic location area or region, and if a geographical location of the mobile computer device 102 of the person 103 enters the geographic location region, then the caregiving computer device 112 and/or the caregiving AI model component 154 may determine a message or notification that may be transmitted to the caregiver computer device 104. Geolocation may be related to, or associated with, the person 103 attending appointment, e.g., as marked on a calendar 152 of the person 103, and likewise, the caregiving computer device 112 may simultaneously track the transactions of the person 103, e.g., to determine that the person 103 also paid a co-pay while at the geolocation of the appointment.

In some embodiments, the caregiving computer device 112 and/or the caregiving AI model component 154 may generate one or more rules or actions that will be performed if one or more criteria is satisfied. In some alternative embodiments, the rules or actions may be set, or requested, by the caregiver 106. The caregiving computer device 112 may execute the rules or actions, automatically, after the caregiving computer device 112 determines that the criteria is satisfied. For example, if a criteria is satisfied, transaction data of the person 103, and/or calendar data 152, is automatically applied to the caregiving AI model component 154 to generate model outputs, such as a notification message that the caregiving computer 112 transmits to the caregiver computer device 104.

In certain embodiments, caregiving system computer device 112 also receives and/or retrieves a personal transaction record 134 from payment processing network 110. Personal transaction record 134 includes a transaction parameter associated with a payment transaction initiated by person 103. Personal transaction record 134 may be transmitted to caregiving system computer device 112, in real-time or near real-time, i.e., personal transaction record 134 may be transmitted to caregiving system computer device 112 by payment processing network 110 at the same or approximately the same time as when person 103 initiated the payment transaction. In other examples, receipt or retrieval of personal transaction record 134 may be delayed from the initiation of a payment transaction such that personal transaction records 134 may be retrieved periodically in a batch, e.g., via a query submitted by caregiving system computer device 112 to transaction database 114.

Caregiving system computer device 112 may receive a plurality of tracking messages 132 from person 103 and/or a plurality of personal transaction records 134 from payment processing network 110. In some examples, caregiving system computer device 112 may receive/retrieve tracking message 132 and personal transaction record 134 regularly or with a predetermined frequency. In other examples, caregiving system computer device 112 may receive and/or retrieve tracking messages 132 and/or personal transaction records 134 using a trigger criteria. For example, the trigger criteria may be every instance a payment transaction is initiated by person 103. In other words, caregiving system computer device 112 may receive personal transaction record 134 each time a payment transaction is initiated by person 103. Additionally, or alternatively, the trigger criteria may be each instance a payment transaction is initiated by person 103 with a specific merchant 105. In other words, personal transaction record 134 may be transmitted only when a payment transaction is initiated with specific service providers 108. For example, the trigger criteria m payment transactions initiated with a health care service provider 108, such that each time that person 103 initiates a purchase transaction with the healthcare service provider, a personal transaction record 134 will be transmitted to caregiving system computer device 112.

Tracking messages 132 and personal transaction records 134 include a set of parameters (tracking parameters and transaction parameters, respectively) being monitored or tracked by caregiving system computer device 112. In other words, caregiving system computer device 112 monitors the tracking parameters and/or the transaction parameters by sampling, saving, and/or calculating data contained in tracking messages 132 and personal transaction records 134. For example, caregiving system computer device 112 may use the tracking parameters contained in the tracking message 132 to monitor the geographical location of person 103, i.e., caregiving system computer device 112 may periodically monitor the location of person 103 from data obtained from Global Positioning system (GPS) data contained in tracking message 132. In other examples, caregiving system computer device 112 may monitor the number and/or timing of payment transactions occurring with a healthcare provider, i.e., caregiving system computer device 112 may count the number of personal transaction records 134 for payment transactions initiated with a healthcare service provider. Additionally, or alternatively, caregiving system computer device 112 may determine and/or monitor any other suitable parameters that enable the caregiving system computer device 112 to function as described herein.

In some embodiments, caregiving system computer device 112 may determine if the monitored transaction parameters and/or tracking parameters satisfy a criteria, more particularly a transaction alert criteria or a tracking alert criteria, respectively. Caregiving system computer device 112 may be configured to determine a need of person 103 in response to a monitored transaction parameter and/or tracking parameter satisfying the respective transaction alert criteria and/or the tracking alert criteria. For example, the transaction parameter may be an amount per transaction and the transaction alert criteria may be an upper limit amount. In other words, caregiving system computer device 112 may monitor amounts of individual transactions initiated by person 103 to determine if the amount per transaction exceeds the upper limit. If person 103 initiates a purchase transaction exceeding the upper limit, thereby satisfying the transaction alert criteria, then caregiving system computer device 112 may determine a need of the person. In some cases, the tracking parameter alert criteria and the transaction parameter alert criteria may be used together to determine a need of the person. In response to caregiving system 100 determining the needs of person 103, caregiving system computer device 112 may transmit a notification message 140 to at least one of caregiver 106 and person 103. Notification message 140 includes information that may be used to indicate to person 103 and/or caregiver 106 the need of the person.

To further illustrate the transaction alert criteria and the tracking alert criteria, an example is provided. Caregiving system computer device 112 may monitor a tracking parameter including scheduling data associated with person 103. The scheduling data may be obtained from, for example, personal care record 125 contained in the personal care database 115. The scheduling data may be used to determine that person 103 has a medical appointment with a healthcare service provider for a specific data and time. In this first example, monitoring the transaction parameters may include monitoring the transactions of person 103 to detect transactions between person 103 and the merchant ID associated with the healthcare provider. In addition, the transaction parameter alert criteria may include confirming that a payment transaction was initiated by person 103 with the healthcare service provider on the specific date for when the medical appointment is scheduled. In other words, if person 103 attends the appointment and pays a copayment, then this may serve as an indication that person 103 has attended their appointment. If no payment transaction is detected between person 103 and the healthcare service provider on the specific date, the transaction parameter alert criteria is satisfied and caregiving system 100 presumes that the person 103 has failed to attend the appointment. In response, caregiving system computer device 112 may determine of the need of person 103 includes rescheduling their appointment. Caregiving system computer device 112 may also transmit notification message 140 to the caregiver, indicating that person 103 may not have attended their appointment and potentially will need to reschedule.

The quantity and types of monitored tracking parameters and/or transaction parameters may be determined by caregiving system 100. Likewise, the tracking parameter alert criteria and the tracking alert criteria may be determined by the caregiving system 100. Additionally, and/or alternatively, person 103 and/or caregiver 106 may select and/or request the quantity and types of monitored tracking parameters, transaction parameters, and the associated tracking parameter alert criteria and/or transaction parameter alert criteria. To further illustrate this, another example is provided. During the registration process, caregiver 106 may request that caregiving system computer system 112 monitor whether person 103 makes a significant purchase, e.g., person 103 spends an amount over $500. As such, caregiving system computer device 112 may monitor the transaction parameters including the transaction amount for each personal transaction record 134. Caregiving system computer device 112 may set the transaction parameter alert criteria including payment transactions exceeding $500.00, such that when person 103 initiates a payment transaction exceeding $500.00, caregiving system computer device 112 may transmit notification message 140 to caregiver 106 indicating that person 103 has made a significant purchase. In other words, in the event that person 103 makes a purchase transaction of an amount exceeding $500.00, for example $501.00, then caregiving system computer device 112 transmits notification message 140 to caregiver 106. Additionally, or alternatively, notification message 140 may include additional details associated with data contained in the personal transaction record 134, for example and without limitation, information about merchant 105 at which the purchase transaction is taking place.

Another example is provided to further illustrate the functionality of caregiving system 100. In some embodiments, caregiver 106 may wish to confirm that person 103 has paid a monthly bill, for example and without limitation, rent, utilities, and the like. As such, caregiving system computer system 112 may track a recurring payment transaction, i.e., a payment transaction occurring each month with a specific service provider 108 for a specific amount and/or a specific range of amounts. For example, caregiver 106 may wish to confirm that person 103 has paid the utility bill each month within a range of values of $75.00 to $200.00. Caregiver 106 may request to be notified if the payment amount of the monthly utility bill exceeds the upper range of values, i.e., payment transaction initiated with a utility service provider exceeds $200.00. Caregiving system computer system 112 may monitor the personal transaction records 134 of the person 103 for transactions having the transaction parameter of merchant ID matching the utility service provider. Caregiving system computer device 112 may set the transaction alert criteria to include personal payment record 134 with the utility service provider that exceeds $200.00. In other words, if person 103 initiates a payment transaction which exceeds $200.00 with the utility service provider, then caregiving system computer device 112 may transmit notification message 140 to caregiver 106 indicating that person 103 is initiating a purchase transaction exceeding $200.00 with the utility service provider.

In certain embodiments, caregiving system computer device 112 may determine a need associated with person 103. Caregiving system computer device 112 may perform a look-up operation to retrieve service provider record 126 stored in-service provider database 116 based on the need determined by caregiving system computer device 112. Caregiving system computer device 112 may then transmit a recommendation message 142 to at least one of person 103 and/or caregiver 106. After the need of person 103 has been determined, then caregiving system computer device 112 may identify service provider 108 that may be hired to complete a task associated with the need of the person.

For example, person 103 and/or caregiver 106 may periodically hire a landscaping company to groom/mow the lawn of the residence associated with person 103. In some cases, person 103 may be neglectful and/or forgetful to hire the landscaping company to mow the lawn. For this example, the transaction parameter may be personal transaction records 134 having the merchant ID matching the landscaping service provider, and the transaction parameter alert criteria may include a threshold period of time over which no personal transaction records 134 are associated with the landscaping service provider. In other words, if person 103 neglects to hire the lawn care service provider to mow the lawn for an extended period of time, then caregiving system computer device 112 may determine that the need of the person includes mowing the lawn of the residence of person 103. As such, caregiving system computer device 112 performs a look-up operation to retrieve service provider record 126 associated with the landscaping service provider from service provider database 116. Caregiving system computer device may transmit a referral message to person 103 and/or caregiver 106 indicating that the lawn at the residence of person 103 may need to be mowed and identifying the service provider 108 that may be hired to mow the lawn. In some examples, service provider database 116 may not include a record 126 for a landscape service, and person 103 and/or caregiver 106 may transmit a query message to the caregiving system computer device 112. The query message may include a request for a recommendation for service provider 108 that may be hired to address a need of person 103, and caregiving system computer device 112 may generate the recommendation as described above.

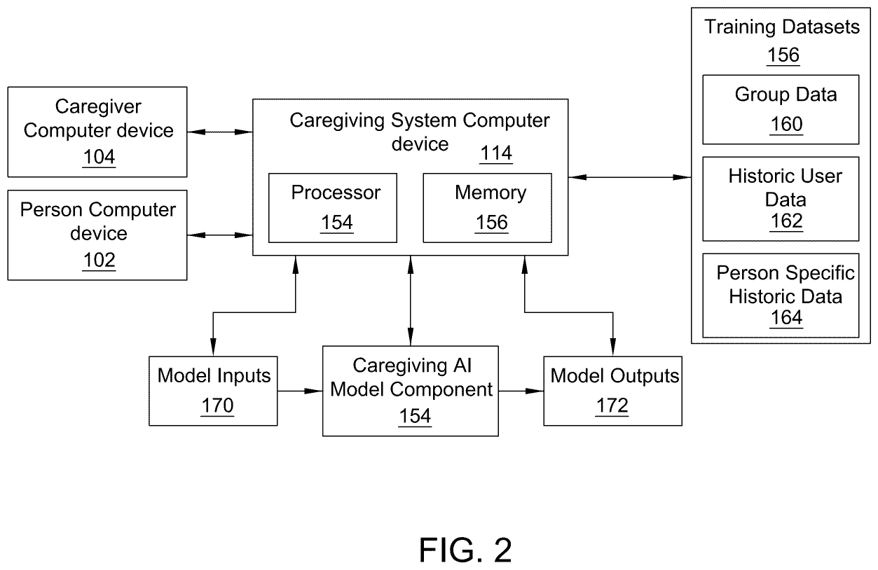

FIG. 2 is a data flow diagram for use with the caregiving system 100 shown in FIG. 1. Caregiving system computer device 112 includes at least one of a processor 154 and at least one of a memory 156. Caregiving system computer device 112 is connected to, or associated with, a caregiving AI model component 154. Caregiving system computer device 112 may build one or more training datasets 156 that the caregiving system computer device 112 may use to train, tune, and/or re-train caregiving AI model component 154. Training datasets 156 may include group data 160, historical user data 162, and/or person specific data 164.

Group data 160 may refer to data associated with a group including a plurality of different people or members. A group may refer to a demographic group having members with similar demographics, e.g., similar residential locations, similar ages, etc. Group data 160 may include holidays (e.g., federally recognized holidays), events (e.g., Superbowl or other sporting events, local festivals, graduations, etc.), a time of year and/or the season. Group data 160 may also refer to financial transactions for groups having similar or related demographics, for example and without limitation, average monthly expenditure, or average expenditure per transaction for a particular MCC. In some embodiments, the person may be included with or associated with the group of the group data 160.

Historical user data 162 is associated with a plurality of different historical users. Each historical record may include historical transaction data, historical user data, and/or historical sensor data. Each historical record may include a historical identified need of the person. The historically identified need of the person may be an identified need as determined by a historical caregiver of the historical user. Additionally, and/or alternatively, the historically identified need of the person may have been previously identified by the model and/or confirmed as a need of the person by the person and/or the caregiver.

Person specific historical data 164 may refer to any historical data associated with the person, e.g., transaction data including having an account identifier of an account of the person, sensor data collected from the person computer device, and/or calendar data 152. Person specific historical data 164 may include demographic data such as age, occupation, or residential location. Person specific historical data 164 may include medical data such as medical health issues or conditions.

Caregiving system computer device 112 may apply one or more model inputs 170 to the caregiving AI model component 154 to generate one or more model outputs 172. Model inputs 170 may include person data, e.g., current person data, including payment transaction data having an account identifier associated with the payment account of the person, sensor data 150, and/or calendar data 152. In some embodiments, person data may include an account balance of the payment account of the person. In some embodiments, person data may include demographic data of the person, medical conditions and/or medications. In some embodiments, caregiving system computer device 112 may apply person response messages and or caregiver response messages to the caregiving AI model component 154.

In some embodiments, model inputs 170 or model training data may include historical merchant transaction data associated with a merchant 105 or service provider 108, such as chargebacks, refunds, average transaction amounts, number or frequency of transactions, transaction history (e.g., providing an indication of how long the merchant 105 and/or service provider 108 has been operating or been in business), a MCC code or frequently used MCC code, and/or any other suitable data associated with transaction occurring at the merchant 105 and/or with the service provider 108. This merchant transaction data may be utilized by the caregiving AI model component 154, e.g., during training, such that a model output 172 may include a ranking of a merchant 105 and/or a service provider 108.

Model outputs 172 may include one or more identified needs of the person. In some embodiments, model outputs 172 may include one or more identified service providers capable of addressing the identified need of the person. In some embodiments model outputs include a severity score associated with each of the one or more identified needs of the person. In some embodiments, model outputs 172 may include a ranking for each of the one or more identified service providers. In some embodiments, model outputs 172 may include an identification of a reoccurring payment, for example a reoccurring payment amount and a reoccurring payment date. In some embodiments, model outputs 172 includes one or more messages to be transmitted to the person computer device 102 and/or caregiver computer device 104.

In some embodiments, caregiving AI model component 154 is trained using the person specific historical data 164, and as such the caregiving AI model component 154 may be used to determine a person profile representative of the normal and/or acceptable behavioral patterns of the person. The person profile may be associated with behaviors that the person previously performed, for example, the person profile may include a transaction amount for transactions occurring at a merchant having a specific MCC code. Additionally, and/or alternatively, the person profile may be associated with behaviors that the person has not previously performed but are not considered impossible and/or resulting in an identified need of the person, for example, the person profile may include, an average and standard deviation for transaction amounts for a transactions occurring at a merchant having a specific MCC code.

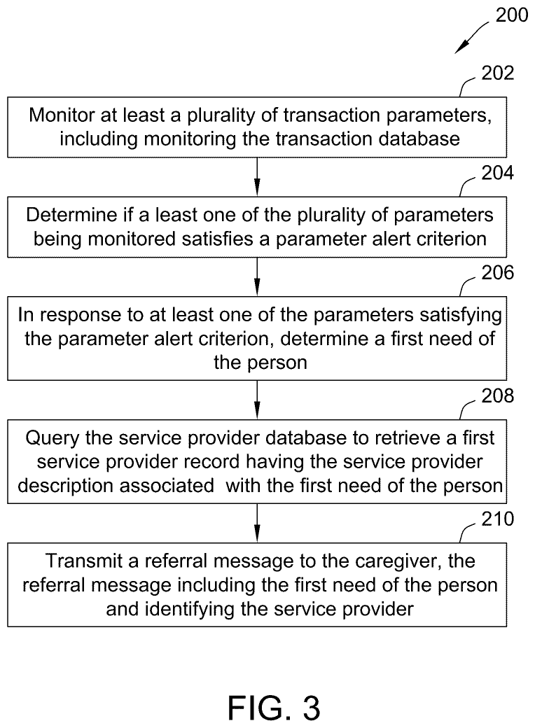

FIG. 3 is a process flow diagram of a method 200 for monitoring the needs of a person (e.g., person 103) and for identifying a service provider (e.g., service provider 108) that may be hired by at least one of the person or a caregiver (e.g., caregiver 106, and/or person 103). In the example embodiment, method 200 is implemented by a caregiving system computer device (e.g., caregiving system computer device 112). The caregiving system computer device includes at least one processor 154 (shown in FIG. 2). The caregiving system computer device is communicatively coupled to a transaction database (e.g., transaction database 114) and a service provider database (e.g., service provider database 116). In some embodiments, the caregiving system computer device is also communicatively coupled to a personal care database (e.g., personal care database 115).

The transaction database stores a plurality of transaction records (e.g., transaction records 122) associated with a plurality of transactions each initiated by one of a plurality of account holders at one of a plurality of merchants (e.g., merchants 105), wherein each transaction record includes a merchant identifier associated with the respective merchant and an account identifier associated with the respective account holder, and wherein at least one of the account holders is the person.

The service provider database stores a plurality of service provider records (e.g., service provider records 126) each associated with one of a plurality of service providers, wherein each service provider record includes a service provider identifier, a service provider contact data, and a service provider description associated with the types of goods and/or service provided by the service provider, wherein the service provider description is associated with a need of the person. For example, the service provider identifier may be the merchant ID used by payment processing network 110, or another unique identifier assigned by caregiving system computer device 112.

The personal care database stores a plurality of personal care records (e.g., personal care records 125), each personal care record including a receiver identifier associated with one of a plurality of care receivers, an item identifier associated with one of a plurality of goods or services required by the respective care receiver, and a schedule data identifying a schedule on which the respective good or service is required, wherein at least one of the plurality of care receivers is the person 103.

In some embodiments, at least one of the transaction database, the service provider database, and/or the personal care database may be filtered using a filtering criteria. Filtering the transaction database may be used to decrease or limit the total number of transaction records in the transaction database that will be considered in a given database operation. Likewise, filtering the service provider database may be used to decrease or limit the total number of service providers in the service provider database that will be considered in a given database operation. Further, filtering the personal care database may be used to decrease or limit the total number of personal care records that will be considered in a given database operation.

Method 200 includes the at least one processor monitoring 202 a plurality of transaction parameters of the person, as discussed above. Monitoring 202 the plurality of transaction parameters includes monitoring the transaction database for transaction records including the account identifier associated with the person. In some embodiments, a payment processing network (e.g., payment processing network 110) may transmit a personal transaction record (e.g., personal transaction record 134) associated with a payment transaction initiated by the person to the caregiving system computer device, which stores the received records in transaction database 114 maintained by the caregiving system computer device. Additionally, or alternatively, the caregiving system computer device has access to transaction database 114 associated with the payment processing network.

In some embodiments, the at least one processor may monitor a plurality of tracking parameters of the person, as discussed above. Monitoring the plurality of tracking parameter of the person may include the at least one processor retrieving a plurality of tracking messages (e.g., tracking messages 132) from a device associated with the person. The device associated with the person may include a personal computer device, for example and without limitation, a laptop, a mobile phone, a tablet, etc. In other example embodiments, the device associated with the person may include a medical device, for example and without limitation, a pacemaker, a heart rate monitor, a wearable biometric tracking device (i.e., set counter, a Fitbit, and the like) and/or a glucose monitoring system. The tracking messages obtained from the device includes a plurality of tracking parameters, for example and without limitation, the geographical location associated with the person, the heart rate associated with the person, and/or a personal schedule (i.e., dates and times of appointments). In other words, the at least one processor may retrieve a plurality of tracking messages from one or more devices associated with the person to monitor a plurality of tracking parameters of the person. For example, method 200 may include monitoring the blood sugar levels of the person, as such the at least one processor may retrieve a tracking message from a glucose monitoring device of the person. In this example, the tracking messages may include the blood sugar level of the person. In yet another example, method 200 may include tracking the location of the person, as such, the at least one processor may retrieve a tracking message from the mobile device associated with the person. In this example, the tracking message may include the Global Positioning coordinates of the mobile device.

Method 200 further includes the at least one processor determining 204 if at least one of the plurality of transaction parameters being monitored satisfies a transaction parameter alert criteria. In response to the transaction parameter satisfying the transaction parameter alert criteria, the at least one processor determines 206 a need of the person. In other embodiments, method 200 further includes, the at least one processor determining if the tracking parameter satisfies a tracking parameter alert criteria. In response to the tracking parameter satisfying the tracking parameter alert criteria, the at least one processor determines a need of the person. For example, the transaction parameter may include recurring payments associated with rental payments of the person. The transaction parameter alert criteria may be associated with the person failing to pay the rental insurance, i.e., a missing recurring payment transaction with a property owner associated with the rental property. In this example, the at least one processor may determine that the need of the person is associated with initiating a payment transaction with the property owner of the rental property. In another example, the tracking parameter may be the location of the person, and the tracking alert criteria may be the location of the person exceeding a 100-mile radius from their residential location. In other words, if the person travels a distance greater than a 100 miles from their home, the tracking parameter satisfies the tracking alert criteria and as such, the need of the person may be associated with directions leading from the current location of the person to the location of the person's residence, and/or a transportation service that may transport the person to the residence.

Method 200 further includes querying 208 the service provider database to retrieve a first service provider record having the service provider description associated with the determined need of the person. In other words, after the at least one processor determines the need of the person then the at least one processor will retrieve a service provider from the service provider database that may be hired to aid the person in addressing the identified need. For example, if the need of the person is identified to be associated with a plumbing service, then the at least one processor will retrieve a service provider including a service provider description associated with a plumbing service category. In other examples, the need of the person may be identified as attending an appointment and as such, the at least one processor may retrieve a transportation service provider from the service provider database.

Method 200 further includes the at least one processor transmitting 210 a referral message to the caregiver, the referral message includes the need of the person and the service provider retrieved from the service provider database. In some cases, the at least one processor may transmit the referral message to the person.

In other embodiments, the at least one processor may transmit a notification message (e.g., notification message 140) to the caregiver. The notification message may include the need of the person. In other words, the notification message may indicate the need of the person to the caregiver. In some other cases, the notification message may be associated with a confirmation, i.e., indicating that the needs of the person are addressed. For example, the notification message may include a confirmation message indicating that the person has attended an appointment or paid a recurring payment. In some cases, the notification message may be transmitted to the person.

In some embodiments, tracking parameters and transaction parameters may be used together. In some cases, the transaction parameter alert criteria may be associated with a tracking parameter alert criteria. For example, the tracking parameter may include scheduling data retrieved from the personal care database and/or from tracking messages obtained from a device associated with the person. The transaction parameter alert criteria may be associated with the scheduling data. For example, the person may be scheduled to attend an appointment with a healthcare service provider on a specific date. As such, the transaction parameter may include a transaction initiated by the person with the healthcare service provider on the specific date. The transaction parameter alert criteria may include an absence of any transaction with the healthcare service provider on the specific date. In other words, the payment transaction initiated with the healthcare service provide may serve as an indication that the person has attended an appointment. In this example, in response to the transaction parameter satisfying the transaction parameter criteria, then the at least one processor may determine the need of the person includes rescheduling the appointment with the healthcare service provider. Further, the at least one service provider may transmit a referral message to the caregiver identifying the service provider (i.e., the healthcare provider) and the need to reschedule.

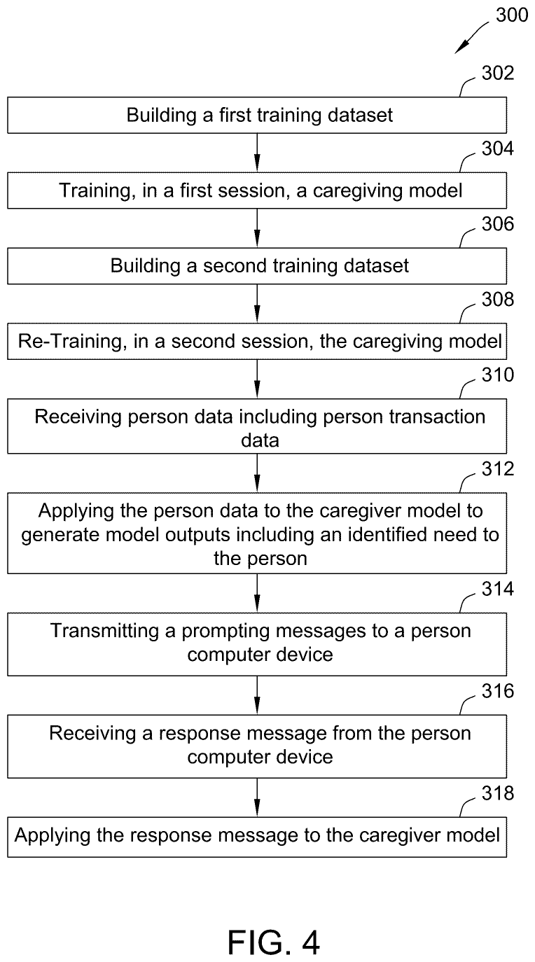

FIG. 4 is a process flow diagram of another method 300 for identifying the needs of a person (e.g., person 103) and for identifying a service provider (e.g., service provider 108) that may be hired by at least one of the person or a caregiver (e.g., caregiver 106, and/or person 103). In the example embodiment, method 300 is implemented by a caregiving system computer device (e.g., caregiving system computer device 112). The caregiving system computer device 112 includes at least one processor 154 (shown in FIG. 2). The caregiving system computer device 112 is communicatively coupled to a transaction database (e.g., transaction database 114), a service provider database (e.g., service provider database 116) and/or a caregiving model (e.g., caregiving AI model component 154). In some embodiments, the caregiving system computer device 112 is also communicatively coupled to a personal care database (e.g., personal care database 115).

Method 300 may include caregiving system computer device 112 building 302 a first training dataset. The first training dataset including a plurality of historical user records. Each historical user record includes historical data associated with a user (e.g., historical transaction data, historical sensor data 150, and historical calendar data 152), a historically identified need of the user, and/or a historical identified service provider.

Method 300 may include caregiving system computer device 112 training 304, in a first session, caregiving AI model component 154 using the first training dataset. The trained caregiving AI model component 154 is enabled to generate one or more model outputs 172 when one or more model inputs 170 are applied. For example, model inputs 170 may include person data and model outputs 172 may include an identified need of the person 102.

Method 300 may include caregiving system computer device 112 collecting recent and/or current person data, e.g., transaction data, sensor data, and/or historical calendar data 152, over a training period of time. The method 300 may include caregiving system computer device 112 building 306 a second training data including the person data collected during the training period of time. In some embodiments, method 300 includes caregiving system computer device 112 building 306 the second training dataset using transaction parameter alert criteria and/or the tracking parameter alert criteria, e.g., received from caregiver computer device 104. In some embodiments, method 300 includes caregiving system computer device 112 building 306 the second training dataset using person data received during enrollment from either caregiver computer device 104 or person computer device 102.

Method 300 may include caregiving system computer device 112 training or re-training 308, in a second session, caregiving AI model component 154 using the second training dataset.

In some embodiments, method 300 includes caregiving system computer device 112 training, in a first session, caregiving AI model component 154 using both the first training dataset and the second training dataset.

In some embodiments, method 300 may include caregiving system computer device 112 including group data 160 to the first and/or second training datasets. Group data 160 may refer to any data associated with a group of people, e.g., a group including person 103. In some embodiments, a group may refer to a demographic group having members, including person 103, with similar demographics, e.g., similar residential locations, similar ages, etc. Group data 160 may include holidays (e.g., federally recognized holidays) or events (e.g., Superbowl or other sporting events, local festivals, graduations, etc.), a time of year and/or the season. Group data 160 may also refer to financial transactions of the group, for example and without limitation, local or regional average monthly expenditures, average expenditures for similar or related demographics, average expenditures per transaction for a particular MCC, etc.

Method 300 includes caregiving system computer device 112 re-training or tuning caregiving AI model component 154 as many times as needed such that the caregiving AI model component 154 is trained using updated person data.

Method 300 may include caregiving system computer device 112 receiving and/or receiving 310 person data in real-time. For example, caregiving system computer device 112 receiving and/or retrieving transaction data, sensor data, and/or calendar data 152. In some embodiments, caregiving system computer device 112 receives and/or retrieves messages from person computer device 102 and/or messages from the caregiver computer device 104, such as enrollment messages or response message.

Method 300 may include caregiving system computer device 112 applying 312 one or more model inputs 170 to trained caregiving AI model component 154. Model inputs 170 may include the receiving person data, e.g., transaction data, sensor data 150, and/or calendar data 152. In some embodiments, model inputs 170 may include transaction parameter alert criteria and/or the tracking parameter alert criteria. Model outputs 172 may include an identified need of the person and/or an identified service provider. In some embodiments, model outputs 172 may include prompting messages, e.g., prompting person to respond, purchase an item, and/or hire an identified service provided. In some embodiments, model outputs 172 may include notification messages, e.g., notifying the caregiver of the identified need and/or the identified service provider. In some embodiments, model outputs 172 may include a severity ranking indicating a severity level of the identified need. In some embodiments, model outputs 172 may include a ranking of the identified service providers. In some embodiments, model outputs may include a recurring purchase, e.g., a recurring purchase amount and date of recurring purchase.

Method 300 may include caregiving system computer device 112 transmitting prompting messages to person computer device. The prompting message including an injury message prompting the user to respond to the message.

Method 300 may include evaluating one or more response messages from person computer device 102. In some embodiments, caregiving system computer device 112 may receive response messages from person computer device 102 responsive to the transmitted response messages. In some embodiments, evaluating includes system computer device comparing response messages to normal and/or acceptable responses. In some embodiments, evaluating includes system computer device applying response message to the trained model to generate a model output including an identified need and/or an updated identified need.

Method 300 may include transmitting one or more notification messages to caregiver computer device 104.

In some embodiments, method 300 includes caregiving system computer device 112 comparing the severity ranking to a severity criteria. If the severity criteria are satisfied, then caregiving system computer device 112 may transmit a notification message to caregiver computer device 104. If the severity criteria are not satisfied, then caregiving system computer device 112 may transmit a prompting message to person computer device 102.

In some embodiments, method 300 may include caregiving computer device receiving or retrieving an account balance of a payment account associated with the person. Method 300 may include caregiving system computer device 112 comparing the account balance to a recurring payment to determine if the person has sufficient funds to pay for the recurring purchase. If caregiving system computer device 112 determines that the account balance is less than the recurring purchase amount, the caregiving computer system transmits a request message to caregiver computer device 104 requesting authorization of transfer of fund from a payment account of the caregiver to a payment account of the person.

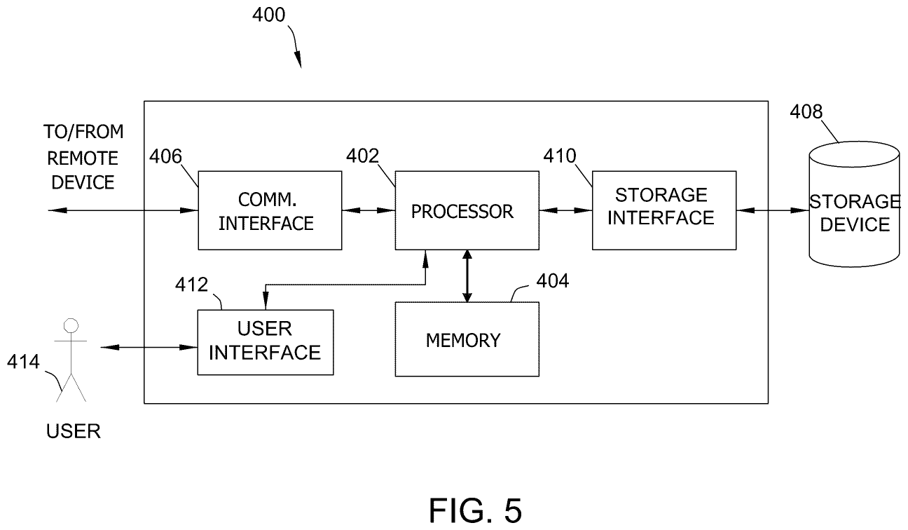

FIG. 5 illustrates an example configuration of a server computer device 400 that may be used to implement caregiving system computer device 112 (shown in FIG. 1). Computer device 400 includes a processor 402 for executing instructions. Instructions may be stored to a memory 404. Processor 402 may include one or more processing units (e.g., in a multi-core configuration) for executing instructions. The instructions may be executed within a variety of different operating systems on data optimizing computer device, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C #, C++, Java, or other suitable programming languages, etc.).

Processor 402 is operatively coupled to a communication interface 406 such that computer device 400 is capable of communication with remote devices. Processor 402 may also be operatively coupled to a storage device 408. For example, storage device 408 is used to implement transaction database 114, service provider database 116, and/or personal care database 115 Storage device 408 is any computer-operated hardware suitable for storing and/or retrieving data. In some embodiments, storage device 408 is integrated in computer device 400. For example, computer device 400 may include one or more hard disk drives as storage device 408. In other embodiments, storage device 408 is external to computer device 400. For example, storage device 408 may include multiple storage units such as hard disks or solid-state disks in a redundant array of inexpensive disks (RAID) configuration. Storage device 408 may include a storage area network (SAN) and/or a network attached storage (NAS) system.

In some embodiments, processor 402 is operatively coupled to storage device 408 via a storage interface 410. Storage interface is any component capable of providing processor 402 with access to storage device 608. Storage interface 410 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 402 with access to storage device 408.

Memory 404 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only and are thus not limiting as to the types of memory usable for storage of a computer program.

Server computer device 400 includes at least one user interface 412 for receiving commands and input from and/or presenting information to a user 414. User interface 412 may, for example, be any component capable of converting and conveying electronic information to and/or from user 414. In some embodiments, user interface 412 includes an output adapter (not shown), such as a video adapter or an audio adapter, which is operatively coupled to processor 402 and operatively coupleable to an output device (also not shown), such as a display device (e.g., a cathode ray tube (CRT), liquid crystal display (LCD), light emitting diode (LED) display, or “electronic ink” display) or an audio output device (e.g., a speaker or headphones). In some embodiments, user interface 412 is configured to include and present a graphical user interface (not shown), such as a web browser or a client application, to user 414. User 412 may display or report via user interface 412, e.g., results generated by one or more of the methods described above. Additionally, or alternatively, server computer device 400 includes an input device (also not shown) for receiving input from user 414. User 414 may use input device, without limitation, to initiate or execute one or more methods or processes described above. Input device may include, for example, a keyboard, a pointing device, a mouse, a stylus, a touch sensitive panel (e.g., a touch pad or a touch screen), a gyroscope, an accelerometer, a position detector, a biometric input device, or an audio input device. A single component such as a touch screen may function as both an output device and input device. Additionally, or alternatively, server computer device 400 is configured to receive commands to execute one or more of the methods described above from, and/or to transmit results of the methods for display to, a remote device via communication interface 406.

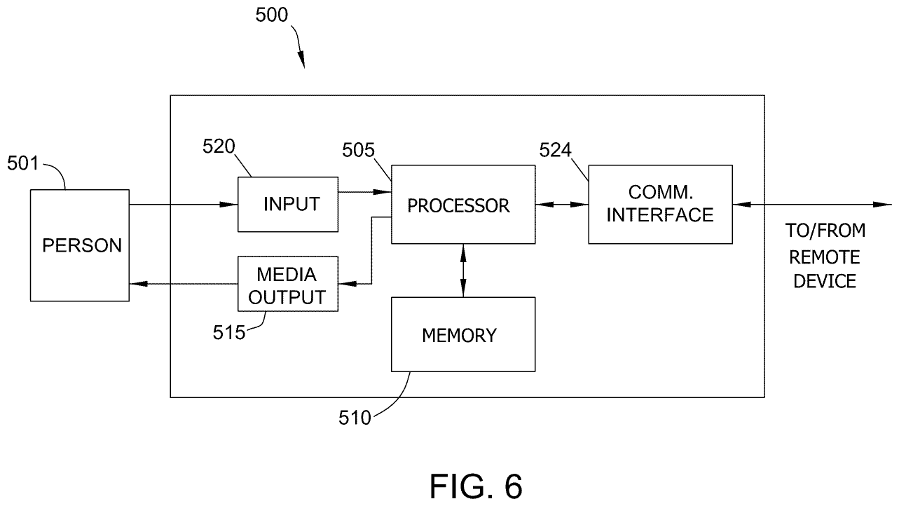

FIG. 6 illustrates an example configuration of a user computer device 500, such as computer device associated with a caregiver (e.g., caregiver 106) and/or a person (e.g., the person 103). The computer device may include laptop, tablet, and/or mobile phone associated with either the person and/or the caregiver. In the example embodiment, user computer device 500, operated by a customer 501 (e.g., the person 103 and/or caregiver 106)). User computer device 500 includes a processor 505 for executing instructions, and a memory 510. In some embodiments, executable instructions are stored in memory 510. Processor 505 may, for example, include one or more processing units (e.g., in a multi-core configuration). Memory 510 may, for example, be any one or more devices allowing information such as executable instructions or transaction data to be stored and retrieved. Memory 510 may further include one or more computer readable media.

In the example embodiment, user computer device 500 further includes at least one media output component 515 for presenting information to customer 501. Media output component 515 may, for example, be any component capable of converting and conveying electronic information to customer 501. In some embodiments, media output component 515 includes an output adapter (not shown), such as a video adapter or an audio adapter, which is operatively coupled to processor 505 and operatively coupleable to an output device (also not shown), such as a display device (e.g., a cathode ray tube (CRT), liquid crystal display (LCD), light emitting diode (LED) display, or “electronic ink” display) or an audio output device (e.g., a speaker or headphones).

In some embodiments, media output component 515 is configured to include and present a graphical user interface such as a web browser or a client application, to customer 501. The graphical user interface may include, for example, an online store interface for viewing or purchasing items, or a wallet application for managing payment information. In some embodiments, user computer device 500 includes an input device 520 for receiving input from customer 501. Customer 501 may use input device 520, without limitation, to select or enter one or more items to purchase or request to purchase, to access credential information, or to access payment information. Input device 520 may include, for example, a keyboard, a pointing device, a mouse, a stylus, a touch sensitive panel (e.g., a touch pad or a touch screen), a gyroscope, an accelerometer, a position detector, a biometric input device, or an audio input device. A single component such as a touch screen may function as both an output device of media output component 515 and input device 520.

In one embodiment, user computer device 500 further includes a communication interface 525, communicatively coupled to a remote device such as server computer system 400 (shown in FIG. 5). Communication interface 525 may include, for example, a wired or wireless network adapter or a wireless data transceiver for use with a mobile telecommunications network.

In the example embodiment, memory 510 stores computer readable instructions for providing a user interface to customer 501 through media output component 515 and, optionally, for receiving and processing input from input device 520. A user interface may include, among other possibilities, a web browser, or a client application. Web browsers enable users, such as customer 501, to display and interact with media and other information typically embedded on a web page or a website from server computer device 400. A client application allows customer 501 to interact with, for example, server computer device 400. For example, instructions may be stored by a cloud service, and the output of the execution of the instructions sent to the media output component 515.

Processor 505 executes computer-executable instructions for implementing aspects of the disclosure. In some embodiments, the processor 505 is transformed into a special purpose microprocessor by executing computer-executable instructions or by otherwise being programmed.

A processor or a processing element may employ artificial intelligence and/or be trained using supervised or unsupervised machine learning, and the machine learning program may employ a neural network, which may be a convolutional neural network, a deep learning neural network, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally, or alternatively, the machine learning programs may be trained by inputting sample data sets or certain data into the programs, such as image data, text data, report data, and/or numerical analysis. The machine learning programs may utilize deep learning algorithms that may be primarily focused on pattern recognition and may be trained after processing multiple examples. The machine learning programs may include Bayesian program learning (BPL), voice recognition and synthesis, image or object recognition, optical character recognition, and/or natural language processing—either individually or in combination. The machine learning programs may also include natural language processing, semantic analysis, automatic reasoning, and/or machine learning.

In supervised machine learning, a processing element may be provided with example inputs and their associated outputs and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs. In one embodiment, machine learning techniques may be used to extract data about the computer device, the user of the computer device, the computer network hosting the computer device, services executing on the computer device, and/or other data.

Based upon these analyses, the processing element may learn how to identify characteristics and patterns that may then be applied to training models, analyzing transaction and authentication data, and detecting and analyzing risk.

The computer-implemented methods discussed herein may include additional, less, or alternate actions, including those discussed elsewhere herein. The methods may be implemented via one or more local or remote processors, transceivers, servers, and/or sensors (such as processors, transceivers, servers, and/or sensors mounted on vehicles or mobile devices, or associated with smart infrastructure or remote servers), and/or via computer-executable instructions stored on non-transitory computer-readable media or medium.

In some embodiments, caregiving computer system is configured to implement machine learning, such that the caregiving computer system “learns” to analyze, organize, and/or process data without being explicitly programmed. Machine learning may be implemented through machine learning methods and algorithms (“ML methods and algorithms”). In an exemplary embodiment, a machine learning module (“ML module”) is configured to implement ML methods and algorithms. In some embodiments, ML methods and algorithms are applied to data inputs and generate machine learning outputs (“ML outputs”). Data inputs may include but are not limited to images, text data, and/or other types of data. ML outputs may include, but are not limited to identified objects, items classifications, textual product, and/or other data extracted from the images or textual data. In some embodiments, data inputs may include certain ML outputs.

In some embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of machine learning, such as supervised learning, unsupervised learning, and reinforcement learning.

In one embodiment, the ML module employs supervised learning, which involves identifying patterns in existing data to make predictions about subsequently received data. Specifically, the ML module is “trained” using training data, which includes example inputs and associated example outputs. Based upon the training data, the ML module may generate a predictive function which maps outputs to inputs and may utilize the predictive function to generate ML outputs based upon data inputs. The example inputs and example outputs of the training data may include any of the data inputs or ML outputs described above. In the exemplary embodiment, a processing element may be trained by providing it with a large sample of text with known characteristics or features. Such information may include, for example, information associated with a plurality of text of a plurality of different vendors, data sources, objects, items, and/or property.

In another embodiment, a ML module may employ unsupervised learning, which involves finding meaningful relationships in unorganized data. Unlike supervised learning, unsupervised learning does not involve user-initiated training based upon example inputs with associated outputs. Rather, in unsupervised learning, the ML module may organize unlabeled data according to a relationship determined by at least one ML method/algorithm employed by the ML module. Unorganized data may include any combination of data inputs and/or ML outputs as described above.

In yet another embodiment, a ML module may employ reinforcement learning, which involves optimizing outputs based upon feedback from a reward signal. Specifically, the ML module may receive a user-defined reward signal definition, receive a data input, utilize a decision-making model to generate a ML output based upon the data input, receive a reward signal based upon the reward signal definition and the ML output, and alter the decision-making model so as to receive a stronger reward signal for subsequently generated ML outputs. Other types of machine learning may also be employed, including deep or combined learning techniques.

In some embodiments, generative artificial intelligence (AI) models (also referred to as generative machine learning (ML) models) may be utilized with the present embodiments and may the voice bots or chatbots discussed herein may be configured to utilize artificial intelligence and/or machine learning techniques. For instance, the voice or chatbot may be a ChatGPT chatbot. The voice or chatbot may employ supervised or unsupervised machine learning techniques, which may be followed by, and/or used in conjunction with, reinforced or reinforcement learning techniques. The voice or chatbot may employ the techniques utilized for ChatGPT. The voice bot, chatbot, ChatGPT-based bot, ChatGPT bot, and/or other bots may generate audible or verbal output, text, or textual output, visual or graphical output, output for use with speakers and/or display screens, and/or other types of output for user and/or other computer or bot consumption.

Based upon these analyses, the processing element may learn how to identify characteristics and patterns that may then be applied to analyzing and classifying objects. The processing element may also learn how to identify attributes of different objects in different lighting. This information may be used to determine which classification models to use and which classifications to provide.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

As will be appreciated based on the foregoing specification, the above-described embodiments of the disclosure may be implemented using computer programming or engineering techniques including computer software, firmware, hardware or any combination or subset thereof, wherein the technical effect or enabling individual chargeback tracking, settlement, and recording. Any such resulting program, having computer-readable code means, may be embodied, or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps,” or code) include machine instructions for a programmable processor and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer system for analyzing data using AI modeling tools to detect anomalous data associated with a person, the computer system comprising:
at least one memory device;
an AI modeling component for storing at least one model configured to identify anomalous data of the person, the at least one model trained using historical user transaction data for a plurality of users that is labeled with user need data; and
at least one processor in communication with the at least one memory device and the AI modeling component, the at least one processor programmed to:
receive person data associated with the person including payment transaction records including at least an account identifier associated with a payment account of the person and a merchant identifier for identifying a merchant involved in the transaction;
input the person data into the at least one AI model to generate one or more outputs including (i) identifying anomalous data of the person, (ii) identifying at least one present need of the person from the anomalous data, and (iii) outputting a recommendation for addressing the at least one present need; and
transmit a notification message to a caregiver computer device associated with a caregiver of the person, the notification message including the identified at least one present need of the person and the outputted recommendation.

2. The computer system of claim 1, wherein the processor is further programmed to:
receive, from the caregiver computer device, at least one of a tracking alert criteria comprising a geographic location area;
if the tracking alert criteria is satisfied, input transaction data into the at least one AI model to generate one or more outputs including an indication of whether the person has initiated a transaction associated with the geographic location area; and
if the person has not initiated the transaction, transmit a notification message to the caregiver computer device.

3. The computer system of claim 1, wherein the processor is further programmed to:
build a first training dataset including a plurality of historical user records associated with a plurality of historical users, wherein each historical user records includes historical transaction data, historical user data, and at least one historical need of the historical user; and
train, in a first training session, the AI model using the first training dataset to generate the trained caregiving model.

4. The computer system of claim 3, wherein the processor is further programmed to:
build a second training dataset including a plurality of historical person records associated with the person, wherein historical person record includes historical transaction data, historical person data, and at least one historical need of the person as previously determined using the AI model; and
re-train, in a second training session, the AI model using the second training dataset to generate the trained AI model.

5. The computer system of claim 1, wherein the processor is further programmed to:
build a first training dataset including a plurality of historical user records and group records, the historical user records are associated with a plurality of historical users, wherein each historical user records includes historical transaction data, historical user data, and at least one historical need of the historical user as previously determine using the AI model, the group records are associated with a group of historical users, wherein group records include an average transaction amount for a plurality of users within the group; and
re-train, in a first training session, the AI model using the first training dataset to generate the trained AI model.

6. The computer system of claim 1, where in the processor is further programmed to:
apply the person data to a trained AI model to generate one or more model outputs, wherein model outputs include an identified need of the person and a severity score associated with each identified need of the person; and
compare the severity score to a severity criteria, when the severity criteria are satisfied, transmit the notification message to the caregiver computer device, and if the severity criteria are not satisfied transmit a prompting message to a person computer device associated with the person.

7. The computer system of claim 1, wherein the processor is further programmed to:
apply the person data to the trained AI model to generate one or more model outputs, wherein model outputs include the identified need of the person and a recurring payment of the person, the recurring payment including a recurring payment deadline and a recurring payment amount;
retrieve a balance in the payment account of the person; and
compare the retrieved balance to the recurring payment amount in advance of the recurring payment deadline, and when the balance is less than the recurring payment amount, transmit a request message to the caregiver computer device, wherein the request message includes an authorization to transfer funds from a payment account of the caregiver to the payment account of the person.

8. The computer system of claim 1, wherein the processor is further programmed to:
receive one or more response messages from a person computer device associated with the person; and
apply the response messages to the trained AI model to generate one or more model outputs, wherein model outputs include an identified updated need of the person and a severity score associated with each identified updated need of the person.

9. The computer system of claim 1, wherein the processor is further programmed to:
receive person data associated with the person being cared for by a caregiver, the person data including sensor data collected by a sensor of a person computer device associated with the person, wherein sensor data includes location data.

10. The computer system of claim 1, wherein the processor is further programmed to:
receive person data associated with a person being cared for by a caregiver, the person data including calendar data from a person computer device associated with the person, wherein calendar data includes an appointment time and an appointment location.

11. A computer-implemented method for analyzing data using AI modeling tools to detect anomalous data associated with a person, the method implemented using a computer device including at least one processor, at least one memory device for storing data, and an AI modeling component for storing at least one model configured to identify the anomalous data of the person, the at least one model trained using historical user transaction data for a plurality of users that is labeled with user need data, the method comprising:
receiving person data associated with the person including payment transaction records including at least an account identifier associated with a payment account of the person and a merchant identifier for identifying a merchant involved in the transaction;
inputting the person data into the at least one AI model to generate one or more outputs including (i) identifying anomalous data of the person, (ii) identifying at least one present need of the person, and (iii) outputting a recommendation for addressing the at least one present need; and
transmitting a notification message to a caregiver computer device associated with a caregiver of the person, the notification message including the identified at least one present need of the person and the outputted recommendation.

12. The method of claim 11, wherein the method further includes:
receiving, from the caregiver computer device, at least one of a tracking alert criteria comprising a geographic location area;
if the tracking alert criteria is satisfied, inputting transaction data into the at least one AI model to generate one or more outputs including an indication of whether the person has initiated a transaction associated with the geographic location area; and
if the person has not initiated the transaction, transmitting a notification message to the caregiver computer device.

13. The method of claim 11, wherein the method further includes:
building a first training dataset including a plurality of historical user records associated with a plurality of historical users, wherein each historical user records includes historical transaction data, historical user data, and at least one historical need of the historical user; and
training, in a first training session, the AI model using the first training dataset to generate the trained caregiving model.

14. The method of claim 11, wherein the method further includes:
building a second training dataset including a plurality of historical person records associated with the person, wherein historical person record includes historical transaction data, historical person data, and at least one historical need of the person as previously determined using the AI model; and
re-training, in a second training session, the AI model using the second training dataset to generate the trained AI model.

15. The method of claim 11, wherein the method further includes:
building a first training dataset including a plurality of historical user records and group records, the historical user records are associated with a plurality of historical users, wherein each historical user records includes historical transaction data, historical user data, and at least one historical need of the historical user as previously determine using the AI model, the group records are associated with a group of historical users, wherein group records include an average transaction amount for a plurality of users within the group; and
re-training, in a first training session, the AI model using the first training dataset to generate the trained AI model.

16. The method of claim 11, wherein the method further includes:
applying the person data to a trained AI model to generate one or more model outputs, wherein model outputs include an identified need of the person and a severity score associated with each identified need of the person; and
comparing the severity score to a severity criteria, when the severity criteria is satisfied, transmit the notification message to the caregiver computer device, and if the severity criteria is not satisfied transmit a prompting message to a person computer device associated with the person.

17. The method of claim 11, wherein the method further includes:
applying the person data to the trained AI model to generate one or more model outputs, wherein model outputs include the identified need of the person and a recurring payment of the person, the recurring payment including a recurring payment deadline and a recurring payment amount;
retrieving a balance in the payment account of the person; and
comparing the retrieved balance to the recurring payment amount in advance of the recurring payment deadline, and when the balance is less than the recurring payment amount, transmit a request message to the caregiver computer device, wherein the request message includes an authorization to transfer funds from a payment account of the caregiver to the payment account of the person.

18. The method of claim 11, wherein the method further includes:
receiving one or more response messages from a person computer device associated with the person; and
applying the response messages to the trained AI model to generate one or more model outputs, wherein model outputs include an identified updated need of the person and a severity score associated with each identified updated need of the person.

19. The method of claim 11, wherein the method further includes:
receiving person data associated with the person being cared for by a caregiver, the person data including sensor data collected by a sensor of a person computer device associated with the person, wherein sensor data includes location data.

20. A non-transitory computer-readable storage medium that includes computer-executable instructions for analyzing data using AI modeling tools to detect anomalous data associated with a person, wherein when executed by a computer device comprising at least one processor, at least one memory device, and an AI modeling component for storing at least one model configured to identify anomalous data of the person, the at least one model trained using historical user transaction data for a plurality of users that is labeled with user need data, when the instructions are executed, the processor is configured to:
receive person data associated with the person including payment transaction records including at least an account identifier associated with a payment account of the person and a merchant identifier for identifying a merchant involved in the transaction;
input the person data into the at least one AI model to generate one or more outputs including (i) identifying anomalous data of the person, (ii) identifying at least one present need of the person from the anomalous data, and (iii) outputting a recommendation for addressing the at least one present need; and
transmit a notification message to a caregiver computer device associated with a caregiver of the person, the notification message including the identified at least one present need of the person and the outputted recommendation.
